(19) United States
(12) Patent Application Publication
(10) Pub. No.: US 2006/0286158 A1
(43) Pub. Date: Dec. 21, 2006

(54) Treatment of overuse tendinopathy using transdermal nitric oxide-generating agents

(76) Inventors: George Anthony Calvert Murrell, Sydney (AU); Robert Ang, New York, NY (US); Ludwig Weimann, Essex Junction, VT (US); Sven Jacobson, New York, NY (US)

Correspondence Address: THORPE NORTH & WESTERN, LLP., 8180 SOUTH 700 EAST, SUITE 200, SANDY, UT, 84070, US

(21) Appl. No.: 11/366,204

(22) Filed: Mar. 1, 2006

Related U.S. Application Data
(63) Continuation-in-part of application No. 10/967,707, filed on Oct. 15, 2004.
(60) Provisional application No. 60/512,070, filed on Oct. 17, 2003.

(51) Int. Cl. A61K 31/21; A61K 9/70

(52) U.S. Cl. 424/449; 514/509

(57) ABSTRACT

The present invention provides methods for treating tendinopathy in the absence of inflammation by the transdermal administration of nitroglycerin. Such methods include methods for relieving pain associated with such tendinopathies, and the use of a transdermal patch configured to deliver glyceryl trinitrate at a rate of 5 mcg/hr to about 85 mcg/hr.

The present application is a continuation-in-part of U.S. patent application Ser. No. 10/967,707, filed Oct. 15, 2004, which claims the benefit of U.S. Provisional Patent Application Ser. No. 60/512,070, filed Oct. 17, 2003, each of which is incorporated herein by reference in their entireties.

### FIELD OF THE INVENTION

The present invention relates to the treatment of overuse tendinopathies, including chronic overuse tendinopathy, using transdermally administered nitric oxide donors.

### BACKGROUND OF THE INVENTION

Overuse Tendinopathies are a common cause of discomfort and pain for many people. There are a number of areas of the body where tendinopathy can occur but some forms are particularly common. Perhaps the most common is Extensor tendinosis. Extensor tendinosis (“tennis elbow” or lateral epicondylitis) is a degenerative overuse tendinopathy of the wrist extensors at their attachment to the lateral humeral epicondyle. No treatment has been universally successful in managing this condition. Although it is frequently referred to as “tennis elbow,” extensor tendinosis is not restricted to tennis players. People at risk of this condition involve those who participate in repetitive upper limb activities involving flexing and extension of the wrist. This includes workers with many occupations such as, for example, carpenters, painters, process workers, and participants in racquet sports, golf, and throwing sports.

Tennis elbow is typically caused by overuse of the tendons which extend the wrist. This causes damage to the tendon at its site of attachment into the elbow. The cellular events that lead to tendon damage are undetermined. Ninety percent of people with tennis elbow develop pain on and around the bony prominence (epicondyle) on the outside (lateral side) of the elbow. The pain is usually exacerbated by activities such as lifting objects, unscrewing jars, playing golf or tennis, and repetitive movements such as painting or hammering nails. In chronic cases, pain may be present with writing and shaking hands and many people describe “aching” of the elbow while at rest.

In addition to tennis elbow, other common degenerative tendinopathies associated with overuse include non-insertional Achilles tendinopathy and rotator cuff tendinopathy. Non-insertional Achilles tendinopathy is especially common among runners, and rotator cuff tendon injury, such as supraspinatus tendinopathy, is prevalent in overhead workers (e.g., painters) and throwing athletes.

There are a variety of non-operative treatments for tendinopathy, many with unproven therapeutic efficacy, and none that are universally effective in the management of chronic tendinopathies. The non-operative management of tendinopathies involves rehabilitation consisting of relative rest, stretching, and a graduated strengthening exercise program focusing on eccentric tendon loading. In some cases, braces can be useful in reducing the force transmitted to the tendon at the joint. Splints to block extension also can be useful by enabling the tendons to rest. Oral anti-inflammatory medications can be useful in some cases and corticosteroid injections can be useful in chronic cases by reducing the pain, thereby enabling a person to perform the rehabilitation exercises.

Relative rest may be a critical aspect of tendon rehabilitation as suggested by recent research on the role of stress activated protein kinases in apoptosis in degenerative tendinopathies. Tendon unloading with heel-raises has been advocated for treating Achilles tendinopathy. Corticosteroid injections remain controversial, and there little evidence that they produce more than a short term therapeutic effect

Nitric oxide (NO) is endogenously produced by three isoforms of the enzyme nitric oxide synthase, inducible nitric oxide synthase (iNOS), an isoform originally found in endothelial cells (eNOS), and an isoform originally found in brain tissue and neuronal cells (bNOS). NO is produced in large amounts by inflammatory cells such as macrophages, neutrophils, lymphocytes and peripheral-blood monocytes during immunological reactions and septic shock. There is also an inducible form of nitric oxide synthase in cartilage.

Wound healing involves the recruitment of inflammatory cells, followed by fibroblasts, to the site of the wound, where collagen and other connective tissue elements are deposited. The collagen fibers then gradually realign to resemble the original connective tissue (e.g., tendon, ligament, skin). Topical NO donation has been used effectively to treat fractures and cutaneous wounds in animal models via mechanisms that may include stimulation of collagen synthesis in fibroblasts. It has been found that that NO modulates collagen synthesis by human tendon fibroblasts in culture. All three isoforms of nitric oxide synthase, the endogenous precursor to NO, are induced during tendon healing. Topical glyceryl trinitrate, a prodrug of NO, has also demonstrated efficacy in improving short term pain in acute supraspinatus tendinitis. Nitric oxide synthase, the endogenous precursor to nitric oxide (NO), is induced during tendon healing and fracture repair.

U.S. Pat. No. 6,190,704 to Murrell, further describes regulation of wound healing by administration of NO or NO generating agents. NO was shown to act as an early initiator of wound healing in soft tissue or tendons in mammals, and administration of agents that increased the concentration of NO in the damaged tissue within the immediate vicinity of the damaged tissue promoted wound healing, e.g., after surgery or trauma. Conversely, administration of agents which decreased the concentration of NO at the site of a wound inhibited wound healing. The latter is useful for conditions where excessive wound healing is detrimental and pathological, such as in arthrofibrosis, Dupuytren's contracture, peritoneal adhesions, frozen shoulder, scleroderma, or keloid formation.

### SUMMARY OF THE INVENTION

The present invention describes the unexpected benefit of glyceryl trinitrate, a topical NO donor, for the treatment of tendinopathy, including pain associated with the condition, using a low concentration of glyceryl trinitrate, e.g., approximately ¼ of that marketed for cardiovascular use (Nitro-Dur™, Schering-Plough). In addition, the present invention provides a method for treating tendinopathy in a mammal in need of such treatment which includes administering a glycerol nitrate containing transdermal patch to a skin site proximate an affected tendon. The transdermal patch can be configured to deliver glyceryl trinitrate at a rate of from about 5 mcg/hr to about 85 mcg/hr.

In one embodiment, a method of relieving pain caused by or associated with overuse tendinopathy in a mammal in need of such treatment can include administering glyceryl trinitrate to a skin site proximate an affected tendon using a transdermal patch. The transdermal patch can be configured to deliver glyceryl trinitrate at a rate of from about 5 mcg/hr to about 85 mcg/hr and the administration can be for a length of time sufficient to relieve the pain caused by the affected tendon.

In another embodiment, a method of reducing tenderness related to overuse tendinopathy in a mammal in need of such treatment can include administering glyceryl trinitrate to a skin site proximate an affected tendon using a transdermal patch. The transdermal patch can be configured to deliver glyceryl trinitrate at a rate of from about 5 mcg/hr to about 85 mcg/hr and the administration can be for a length of time sufficient to reduce the tenderness of the affected tendon.

In a further embodiment, a method of treating overuse tendinopathy in a mammal in need of such treatment can include administering glyceryl trinitrate to a skin site proximate an affected tendon of said mammal using a transdermal patch. The transdermal patch being configured to deliver glyceryl trinitrate at a rate of from about 5 mcg/hr to about 85 mcg/hr. The administration can be for a length of time such that the function of the affected tendon is improved. In still a further embodiment the administration can be continuous until the function of the affected tendon is improved.

In yet another embodiment, a method of treating overuse tendinopathy in a mammal in need of such treatment which can include administering glyceryl trinitrate to a skin site proximate an affected tendon using a transdermal patch. The transdermal patch can be configured to deliver glyceryl trinitrate at a rate of from about 5 mcg/hr to about 85 mcg/hr. The administration of the transdermal patch can be such that there is decreased pain upon activity, decreased night pain, decreased pain while the tendon is at rest, or combinations thereof.

The present invention also provides a method of relieving pain caused by tendinopathy by transdermally administering glycerol trinitrate or another NO generating agent to the affected tendon.

The present invention also provides combination therapy for the treatment of tendinopathy by transdermally administering glyceryl trinitrate or another nitric oxide-generating agent to the affected tendon and providing a rehabilitation regimen which includes, but is not limited to rest, tendon unloading, orthotics or braces, prolonged daily stretching, or a graduated exercise strengthening program, or combinations thereof. The rehabilitative therapy can be provided for all, or a portion of, the period that the patient is being treated with the NO generating agent.

### BRIEF DESCRIPTION OF THE DRAWINGS

FIG. 1

1(a) depicts the effects of glyceryl trinitrate 1.25 mg/day (˜52 mcg/hr) via transdermal patch plus rehabilitation (GTN, n=41) versus rehabilitation alone (placebo, n=43) on Achilles tendon pain with activity. Statistically significant differences between groups are shown with an asterisk (*p<0.05).

1(b) depicts effects of glyceryl trinitrate 1.25 mg/day (˜52 mcg/hr) via transdermal patch plus rehabilitation (GTN, n=47) versus rehabilitation alone (placebo, n=48) on lateral elbow pain with activity in extensor tendinopathy. Statistically significant differences between groups are shown with an asterisk (*p<0.05).

1(c) shows effects of glyceryl trinitrate 1.25 mg/day (˜52 mcg/hr) via transdermal patch plus rehabilitation (GTN, n=28) versus rehabilitation alone (placebo, n=29) on shoulder pain with activity in supraspinatus tendinopathy. Statistically significant differences between groups are shown with an asterisk (*p<0.05).

FIG. 2

2(a) shows effects of glyceryl trinitrate (GTN, n=41) 1.25 mg/day (˜52 mcg/hr) via transdermal patch, plus rehabilitation versus rehabilitation alone (placebo, n=43), on ORI-ASTS measured ankle plantarflexor mean total work (Achilles tendinopathy). These results are expressed as increases from baseline as there was a significant difference in mean total work at week 0. Statistically significant differences between groups are shown with an asterisk (*p<0.05).

2(b) shows effects of glyceryl trinitrate (GTN, n=47) 1.25 mg/day (˜52 mcg/hr) via transdermal patch, plus rehabilitation versus rehabilitation alone (placebo, n=48), on ORI-TETS measured mean total work (tennis elbow). Statistically significant differences between groups are shown with an asterisk (*p<0.05).

2(c) shows effects of glyceryl trinitrate (GTN, n=28) 1.25 mg/day (˜52 mcg/hr) via transdermal patch plus rehabilitation, versus rehabilitation alone (placebo, n=29), on dynamometer measured supraspinatus force (supraspinatus tendinopathy). Statistically significant differences between groups are shown with an asterisk (*p<0.05, **p<0.01).

FIG. 3

3(a) depicts effects of glyceryl trinitrate (GTN, n=41) 1.25 mg/day (−52 mcg/hr) via transdermal patch plus rehabilitation versus rehabilitation alone (placebo, n=43) on pain scores after the 10 hop test (Achilles tendonitis). Statistically significant differences between groups are shown with an asterisk (*p<0.05, **p<0.01).

3(b) shows effects of glyceryl trinitrate (GTN, n=28) 1.25 mg/day (˜52 mcg/hr) via transdermal patch versus rehabilitation alone (placebo, n=29) on shoulder impingement in internal rotation. Statistically significant differences are shown with an asterisk (*p<0.05).

3(c) demonstrates effects of glyceryl trinitrate (GTN, n=28) 1.25 mg/day (˜52 mcg/hr) via transdermal patch versus rehabilitation alone (placebo, n=29) on passive shoulder abduction range of motion. Statistically significant differences are shown with an asterisk (*p<0.05).

FIG. 4

4(a) shows the percentage differences in mean grouped outcome measures between the glyceryl trinitrate group (GTN 1.25 mg/day patch (˜52 mcg/hr), n=41) and the placebo patch group (n=43); a between group comparison of means for grouped outcome measures in the Achilles tendinopathy clinical trial.

4(b) shows the percentage differences in mean grouped outcome measures between the glyceryl trinitrate group (GTN 1.25 mg/day patch (˜52 mcg/hr), n=47) and the placebo patch group (n=48); a between group comparison of means for grouped outcome measures; a between group comparison of means for grouped outcome measures in the extensor tendinopathy clinical trial.

4(c) shows the percentage differences in mean grouped outcome measures between the glyceryl trinitrate group (GTN 1.25 mg/day patch (˜52 mcg/hr), n=28) and the placebo patch group (n=29); a between group comparison of means for grouped outcome measures; a between group comparison of means for grouped outcome measures in the supraspinatus tendinopathy clinical trial.

### DETAILED DESCRIPTION OF THE PREFERRED EMBODIMENT(S)

The present invention provides a treatment for tendinopathy in the absence of inflammation, especially chronic tendinopathy, which comprises administering an effective amount of glyceryl trinitrate or other NO generating compound, via a transdermal patch. The patch is placed directly on a skin surface that is proximate the affected tendon, and can be replaced periodically over a sufficient period of time to improve force and functional outcome measures at the affected tendon, and/or to relieve pain. In one embodiment the patch is replaced daily (every 24 hours). In another embodiment, a new or replacement patch is placed on a different or new skin site which is also proximate the affected tendon. The present invention exemplifies treating three different chronic overuse tendinopathies using a transdermal patch delivering a significantly less glyceryl trinitrate than the patch that is marketed and indicated for the treatment of angina.

Definitions

Glyceryl trinitrate refers to 1,2,3-trinitroglycerin, 1,2,3-propanetriol trinitrate, or nitroglycerin, CAS No. 55-63-0 (GTN).

Other NO-releasing agents in addition to glyceryl trinitrate that are contemplated for use in the method of the present invention include sodium nitroprusside, N-(Ethoxycarbonyl)-3-(4-morpholinyl)sydnonimine (Molsidomire); 3-morpholinosydnonimine (SIN-1); 1,2,3,4-Oxatriazolium, 5-amino-3-(3,4-dichlorophneyl)-chloride (GEA 3162); 1,2,3,4-Oxatriazolium, 5-amino-3-(3-chloro-2-methyl-phenyl)chloride (GEA50-24); 1,2,3,4-Oxatriazolium,3-(3-chloro-2-methylphenyl)-5-[[[cyanomethylami-no]carbonyl]amino]-hydroxide inner salt (GEA5583); S-nitroso-N-acetyl-D,L-penicillamine (SNAP); 1-[(4′,5′-Bis(carboxymethoxy)-2′-nitrophenyl)methoxy]-2-oxo-3,3,diethyl-1-triazene dipotassium salt (CNO-4); and [1-(4′,5′-Bis(carboxymethoxy)-2′-nitrophenyl)methoxy]-2-oxo-3,3-diethyl-1-triazine diacetoxymethyl ester (CNO-5), all of which are available from Alexis Corp. (San Diego, Calif.). Additional compounds include diethylamine-NO (DEA/NO), IPA/NO, sperinine-NO (SPER/NO), sulfite-NO (SULFI/NO), OXI/NO, and DETA/NO.

As used herein, the term “affected tendon” refers to a tendon that is characterized by pain or tenderness in the absence of inflammation, and is the subject of a diagnosis of tendinopathy according to those skilled in the art, such as described herein. The diagnosis can usually be made by clinical methods e.g., taking a history regarding the problem and examining the patient, and may be aided by soft tissue imaging studies for example, by ultrasound, or MRI. The tendinopathy can be acute or chronic tendinopathy, where “acute” generally means a duration of symptoms days to weeks, and “chronic” generally means a duration of symptoms from months to years.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

A “subject” or “patient” or “mammal” “in need thereof” is an animal that has developed, or is developing acute or chronic tendinopathy, including but not limited to extensor tendinopathy (tennis elbow), Achilles tendinopathy, supraspinatus tend inopathy (rotator cuff), patellar tend inopathy, quadriceps tendinopathy, hip adductor tendinopathy, common flexor tendinopathy of the elbow (golfer's elbow), and tendinopathy of the thumb. The animal is more particularly a mammal, preferably a rodent or a primate, and most preferably a human.

The terms “treat” or “treatment” means to therapeutically intervene in the development of a disease or disorder in a subject showing a symptom of this disease, e.g., tendinopathy. In the context of the present invention, these symptoms can include but are not limited to, pain or tenderness in the affected tendon, limited range of motion or ability to exert a force on the affected tendon without pain, aching of the affected tendon at rest, with activities, and/or at night.

The term “improve function” as used herein means significant increases in force outcome measures at the affected tendon, as determined by routine methods in the art, including but not limited to the Orthopaedic Research Institute-Ankle Strength Testing System (ORI-ASTS), and dynamometer and Tennis Elbow Testing System (ORI-TETS). These tests measure increases in mean total work, and increases in dynamometer resisted force measurements for the affected tendons.

The term “improve function” also means significant increases in functional outcome measures. Function can be determined by, but is not limited to, the 10 hop test for non-insertional Achilles tendinopathy (similar to tests in the newly validated VISA-A Achilles tendon scale), the ORI-TETS mean peak force and mean total work for extensor tendinopathy, and shoulder passive range of motion in abduction and in internal rotation, as well as shoulder impingement in internal rotation rotation and strength as determined by a hand held dynamometer for supraspinatus tendinopathy. Hopping involves Achilles tendon loading through push-off and landing as used in running and jumping; wrist extensor tendon peak force and total work are measured with a modified chair pick-up test (ORI-TETS). Increases in functional outcome also refer to a subject treated according to the method of the present invention becoming asymptomatic with activities of daily living.

The term “relieve pain” means improved patient rated pain scores as determined, for example, using the Mann-Whitney rank sum tests. In the context of the present invention, this also refers to subjective determinations such as decreased tenderness at the affected tendon or joint, decreased night pain at the affected tendon or joint, and decreased pain with activity at the affected tendon or joint.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The terms “continuous” or “continuously” in the context of drug administration refers to a constant, pre-determined amount of drug that is administered over a specified dosing period. A dosing period is the time during which one of the dosage forms in the series is administered to the patient. Accordingly, the dosing regimen will consist of a separate dosing period for administration of each dosage form in the series. Thus, for example, the first dosage form in the series may be worn by the patient for 24 consecutive hours. As one example, as used herein, continuous administration refers to delivery of 1.25 mg of glyceryl trinitrate to a subject over 24 hours via a transdermal patch, for successive 24 hour periods for 12-24 weeks. In this context, continuous administration of the preceding transdermal patch requires replacing the patch every 24 hours.

The term “relative release rate,” “flux rate,” or “delivery rate” is determined from the amount of drug released per unit time from e.g., a transdermal delivery system through the skin and into the bloodstream of a subject. Mean relative release rate may be expressed, e.g., as μg drug/hr or, for comparing delivery systems covering skin areas of different size, as μg drug/cm²/hr. For example, a transdermal delivery system that releases 1.25 mg of glyceryl trinitrate over a time period of 24 hours is considered to have a relative release rate of about 52.1 μg/hr. For purposes of the invention, it is understood that relative release rates may change between any particular time points within a particular dosing interval, and the term therefore only reflects the overall release rate during the particular dosing interval.

Formulations and Administration

Transdermal dosage forms are convenient dosage forms for delivering many different active therapeutically effective agents, including but not limited to glyceryl trinitrate, and the NO donors described above. Transdermal dosage forms are particularly useful for timed release or sustained release of active agents.

Transdermal dosage forms may be classified into transdermal dosage articles and transdermal dosage compositions. The most common transdermal dosage article is a diffusion driven transdermal system (transdermal patch) using either a fluid reservoir or a drug in adhesive matrix system. Transdermal dosage compositions include, but are not limited to, topical gels, lotions, ointments, transmucosal systems and devices, and iontophoretic (electrical diffusion) delivery systems. Preferably, for the method of the present invention, the transdermal dosage form is a transdermal patch. The transdermal dosage form is used in the dosage regimen of the present invention for timed release or sustained release of glyceryl trinitrate.

Transdermal patches used in accordance with the invention preferably include a backing layer made of a pharmaceutically acceptable material which is impermeable to the glyceryl trinitrate. The backing layer preferably serves as a protective cover for the glyceryl trinitrate, and may also provide a support function. Examples of materials suitable for making the backing layer are films of high and low density polyethylene, polypropylene, polyvinylchloride, polyurethane, polyesters such as poly(ethylene phthalate), metal foils, metal foil laminates of such suitable polymer films, textile fabrics, if the components of the reservoir cannot penetrate the fabric due to their physical properties, and the like. Preferably, the materials used for the backing layer are laminates of such polymer films with a metal foil such as aluminum foil. The backing layer can be any appropriate thickness to provide the desired protective and support functions. A suitable thickness will be from about 10 to about 200 microns. Desirable materials and thickness will be apparent to the skilled artisan.

In certain preferred embodiments, the transdermal dosage forms used in accordance with the invention contain a pharmacologically or biologically acceptable polymer matrix layer. Generally, the polymers used to form the polymer matrix are those capable of forming thin walls or coatings through which pharmaceuticals can pass at a controlled rate. A non-limiting list of exemplary materials for inclusion in the polymer matrix includes polyethylene, polypropylene, ethylene/propylene copolymers, ethylene/ethylacrylate copolymers, ethylenevinyl acetate copolymers, silicones, rubber, rubber-like synthetic homo-, co- or block polymers, polyacrylic esters and the copolymers thereof, polyurethanes, polyisobutylene, chlorinated polyethylene, polyvinylchloride, vinyl chloride-vinyl acetate copolymer, polymethacrylate polymer (hydrogel), polyvinylidene chloride, poly(ethylene terephthalate), ethylene-vinyl alcohol copolymer, ethylene-vinyloxyethanol copolymer, silicones including silicone copolymers such as polysiloxane-polymethacrylate copolymers, cellulose polymers (e.g., ethyl cellulose, and cellulose esters), polycarbonates, polytetrafluoroethylene and mixtures thereof. Exemplary materials for inclusion in the polymer matrix layer are silicone elastomers of the general polydimethylsiloxane structures, (e.g., silicone polymers). Preferred silicone polymers cross-link and are pharmaceutically or biologically acceptable.

Other preferred materials for inclusion in the polymer matrix layer include: silicone polymers that are cross-linkable copolymers having dimethyl and/or dimethylvinyl siloxane units that can be crosslinked using a suitable peroxide catalyst. Also preferred are those polymers consisting of block copolymers based on styrene and 1,3-dienes (particularly linear styrene-isoprene-block copolymers of styrene-butadiene-block copolymers), polyisobutylenes, polymers based on acrylate and/or methacrylate.

The polymer matrix layer may optionally include a pharmaceutically acceptable crosslinking agent. A non-limiting example of suitable crosslinking agent is tetrapropoxy silane. Preferred transdermal delivery systems used in accordance with the methods of the present invention include an adhesive layer to affix the dosage form to the skin of the patient for the desired period of administration. If the adhesive layer of the dosage form fails to provide adhesion for the desired period of time, it is possible to maintain contact between the dosage form with the skin by, for instance, affixing the dosage form to the skin of the patient with an adhesive tape, e.g., surgical tape.

The adhesive layer preferably includes using any adhesive known in the art that is pharmaceutically compatible with the dosage form and preferably hypoallergenic, such as polyacrylic adhesive polymers, acrylate copolymers (e.g., polyacrylate) and polyisobutylene adhesive polymers. In other preferred embodiments of the invention, the adhesive is a hypoallergenic and pressure-sensitive contact adhesive.

The transdermal dosage forms that can be used in accordance with the present invention may optionally include a permeation enhancing agent. Permeation enhancing agents are compounds that promote penetration and/or absorption of the NO-generating agent, e.g., glyceryl trinitrate, through the skin or mucosa and into the blood stream of the patient. A non-limiting list of permeation enhancing agents includes polyethylene glycols, surfactants, and the like.

Alternatively, permeation of the active agent such as glyceryl trinitrate may be enhanced by occlusion of the dosage form after application to the desired site on the patient with, e.g. an occlusive bandage. Permeation may also be enhanced by removing hair from the application site by, e.g. clipping, shaving or use of a depilatory agent. Another permeation enhancer is heat. It is thought that permeation can be enhanced by, among other things, the use of a radiating heat form, such as an infrared lamp, at the application site during at least a portion of the time the transdermal dosage form is applied on the skin or mucosa. Other means of enhancing permeation of the active agent, such as the use of iontophoretic means, are also contemplated to be within the scope of the present invention.

The active agent, e.g., glyceryl trinitrate, may be included in the device in a drug reservoir, drug matrix or drug/adhesive layer. This area of the patch, and the amount of active agent per unit area, determine the limit dose, as one of ordinary skill in the art can readily determine.

Certain preferred transdermal delivery systems also include a softening agent in the reservoir or matrix. Suitable softening agents include higher alcohols such as dodecanol, undecanol, octanol, esters of carboxylic acids, wherein the alcohol component may also be a polyethoxylated alcohol, diesters of dicarboxylic acids, such as di-n-butyladiapate, and triglycerides, particularly medium-chain triglycerides of caprylic/caproic acids or coconut oil. Further examples of suitable softeners are, for example, multivalent alcohols such as glycerol and 1,2-propanediol, as well as softeners such as levulinic acid and caprylic acid, which can also be esterified by polyethylene glycols.

Transdermal dosage systems are described further in U.S. Pat. No. 6,231,885 to Carrara; U.S. Pat. No. 5,948,233 to Burton; U.S. Pat. No. 5,324,521 to Gertner; and U.S. Pat. No. 5,310,559 to Shah et al.

Commercially available transdermal glyceryl trinitrate dosage forms include Deponit™ (Schwarz), Minitran™ (3M), Nitro-Dur™ (Schering-Plough), Percutol™ (Dominion), Transiderm-Nitro™ (Novartis), and Trintek™ (Goldschield). For example, the Nitro-Dur™ patch is a transdermal infusion system that provides continuous controlled-release through intact skin. The Nitro-Dur patches come with varying delivery rates ranging from 0.1 mg/hr to 0.8 mg/hr, and such patches can contain from 20 mg of nitroglycerin to 160 mg of nitroglycerin in an acrylic-based polymer adhesive with resinous cross-linking agent to provide continuous administration. The rate of release is linear, depending on the area of the patch, with each cm² of applied patch delivering approximately 0.02 mg per hour. Thus, the patch containing 40 mg patch delivers approximately 0.1 mg/hr over a patch area of 10 cm². Each unit is sealed in a paper polyethylene-foil pouch.

Patches containing glyceryl trinitrate are further described in U.S. Pat. No. 5,762,952 to Barnhart; U.S. Pat. No. 5,613,958 to Kochinke et al.; U.S. Pat. No. 5,252,165 to Govil; and U.S. Pat. No. 4,615,699 to Gale et al.; which are incorporated herein by reference.

In another embodiment, transdermal administration is achieved by liposomes. Lipid bilayer vesicles are closed, fluid-filled microscopic spheres which are formed principally from individual molecules having polar (hydrophilic) and non-polar (lipophilic) portions. The hydrophilic portions may comprise phosphato, glycerylphosphato, carboxy, sulfato, amino, hydroxy, choline or other polar groups. Examples of lipophilic groups are saturated or unsaturated hydrocarbons such as alkyl, alkenyl or other lipid groups. Sterols (e.g., cholesterol) and other pharmaceutically acceptable adjuvants (including antioxidants such as alpha-tocopherol) may also be included to improve vesicle stability or confer other desirable characteristics.

Liposomes are a subset of these bilayer vesicles and are comprised principally of phospholipid molecules that contain two hydrophobic tails consisting of fatty acid chains. Upon exposure to water, these molecules spontaneously align to form spherical, bilayer membranes with the lipophilic ends of the molecules in each layer associated in the center of the membrane and the opposing polar ends forming the respective inner and outer surface of the bilayer membrane(s). Thus, each side of the membrane presents a hydrophilic surface while the interior of the membrane comprises a lipophilic medium. These membranes may be arranged in a series of concentric, spherical membranes separated by thin strata of water, in a manner not dissimilar to the layers of an onion, around an internal aqueous space. These multilamellar vesicles (MLV) can be converted into Unilamellar Vesicles (UV) with the application of a shearing force.

Liposomes, or unhydrated pro-liposomes, can be administered via transdermal patches. See also U.S. Pat. No. 6,312,715 to Cantor et al., which describes a drug delivery composition comprising pressure sensitive adhesive polymeric microspheres.

In addition, the present invention contemplates the use of any topical dosage form known in the art. Such dosage forms include topical solutions, suspensions, ointments, pastes, creams, lotions, gels, and the like. Preparations of such dosage forms are well known in the art and can be formulated using numerous known excipients.

Such pharmaceutically acceptable excipients include polymers, oils, liquid carriers, surfactants, buffers, preservatives, stabilizers, antioxidants, moisturizers, emollients, colorants, odorants, and mixtures thereof.

Examples of pharmaceutically acceptable polymers suitable for such topical formulations include, but are not limited to, acrylic polymers, cellulose derivatives, such as carboxymethylcellulose sodium, methylcellulose or hydroxypropylcellulose; natural polymers, such as alginates, tragacanth, pectin, xanthan, cytosan, and mixtures thereof.

Examples of suitable pharmaceutically acceptable oils which are so useful include but are not limited to, mineral oils, silicone oils, fatty acids, alcohols, glycols, and mixtures thereof.

Examples of suitable pharmaceutically acceptable liquid carriers include, but are not limited to, water, alcohols or glycols such as ethanol, isopropanol, propylene glycol, hexylene glycol, glycerol and polyethylene glycol, and mixtures thereof, or other mixtures in which the pseudopolymorph is dissolved or dispersed, optionally with the addition of non-toxic anionic, cationic or non-ionic surfactants, inorganic or organic buffers, and mixtures thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, parabens (such as methyl paraben, ethyl paraben, propyl paraben, etc.), and mixtures thereof.

Suitable examples of pharmaceutically acceptable stabilizers and antioxidants include, but are not limited to, ethylenediaminetetraacetic acid (EDTA), thiourea, tocopherol, butyl hydroxyanisole, and mixtures thereof.

Suitable examples of pharmaceutically acceptable moisturizers include, but are not limited to, glycerine, sorbitol, urea, polyethylene glycol, and mixtures thereof.

Suitable examples of pharmaceutically acceptable emollients include, but are not limited to, mineral oils, isopropyl myristate, isopropyl palmitate, and mixtures thereof.

The use of dyes and odorants in topical formulations of the present invention depends on many factors including organoleptic acceptability to the population that will be using the pharmaceutical formulations.

Combination Therapy

The dosage forms used in the method of the present invention may be administered alone or in combination with other active agents, e.g., such as an analgesic or anti-inflammatory, including, for example, a non-steroidal anti-inflammatory drug (NSAID) such as acetaminophen, ibuprofen, or acetylsalicylic acid. For combination treatment with more than one active agent, where the active agents are in separate dosage formulations, the active agents can be administered concurrently, or they each can be administered at separately staggered times. The dosage amount may be adjusted when combined with other active agents as described above to achieve desired effects. Alternatively, unit dosage forms of these various active agents may be independently optimized and combined to achieve a synergistic result wherein the pathology is reduced more than it would be if either active agent were used alone.

Dosages

The dosage of the nitric oxide donor, e.g., glyceryl trinitrate, according to the present invention can be determined on an individual, case-by-case basis by one of ordinary skill in the art, but the transdermal patch will not exceed a delivery rate of nitric oxide donor of about 85 mcg/hr. In one embodiment, the transdermal patch will deliver from about 5 mcg/hr to about 85 mcg/hr of an NO donor. In another embodiment, the transdermal patch will deliver from about 15 mcg/hr to about 75 mcg/hr of an NO donor. In a preferred embodiment, the transdermal patch will deliver from about 30 mcg/hr to about 65 mcg/hr of an NO donor.

### EXAMPLES

### Example 1

The following Example demonstrates that the topical nitric oxide donor glyceryl trinitrate, at 1.25 mg/24 hour (about 52.1 mcg/hr), has clinically demonstrated efficacy in modulating pain, force measures, functional measures, and patient outcomes at three and six months in three common chronic overuse tendinopathies.

This example of practicing the invention is understood to be exemplary only, and does not limit the scope of the invention or the appended claims. A person of ordinary skill in the art will appreciate that the invention can be practiced in many forms according to the claims and disclosures herein.

Patients. Three clinical trials were approved by an institutional Ethics Committee. Patients with clinical diagnoses of the specified tendinopathies were recruited through newspaper advertisements and private consulting rooms. All subjects were over 18 years of age, and gave written informed consent.

In the non-insertional Achilles tendinopathy trial, there were 65 patients (84 Achilles tendons) with 40 men and 25 women enrolled in the study, having a median age of 49 years (range 24 to 77 years), and a median duration of symptoms prior to the study of 16 months (range 4-147 months). In the extensor tendinopathy trail there were 86 patients (95 elbows), with 42 males and 44 females, having a median age of 46 years (range 30 to 74 years), and a median duration of symptoms of 17 months (range 3-232 months). In the supraspinatus tendinopathy trial there were 53 patients (57 shoulders), with 24 males and 29 females, having a median age of 52 years (range 25 to 79 years), and a median symptom duration of 14 months (range 4-96). In all trials there were no significant differences between groups with respect to age, sex, affected side, symptom severity, or symptom duration.

Diagnosis. Diagnostic criteria for patient inclusion in the respective trials were as follows: 1) the diagnosis of chronic non-insertional Achilles tendinopathy was based on an insidious onset of Achilles tendon pain, a tender nodule localized to the region 2 to 6 centimeters from the calcaneal insertion, and an ultrasound examination that excluded a frank tendon tear; 2) the diagnosis of chronic extensor tendinopathy at the elbow was based on an insidious onset of lateral elbow pain, tenderness localized to the lateral humeral epicondyle and extensor carpi radialis brevis tendon, pain in the lateral elbow with resisted wrist or third metacarpophalangeal joint extension, and an ultrasound examination that excluded a frank tendon tear, 3) the diagnosis of chronic supraspinatus tendinopathy was based on positive impingement signs (internal or external rotation), pain with supraspinatus muscle testing, and magnetic resonance imaging (MRI) high signal intensity without frank tear in the supraspinatus tendon.

Patients were excluded if they had: tendinopathy of less than three months duration, current pregnancy, previous surgery on the affected limb or tendon, dislocation of the ipsilateral limb joints, distal neurological signs, a local corticosteroid injection in the previous three months, the current use of nitrate medications or phosphodiesterase inhibitors such as Viagra™, a family history of arthritis other than osteoarthritis, or extra-articular features of seronegative arthropathies.

For the three clinical trials, patients with a clinical diagnosis of the respective chronic tendinopathy were recruited through newspaper advertisements and private consulting rooms, and were randomly allocated into two groups. One group performed tendon rehabilitation and used the active transdermal patch (one quarter of a 5 mg/24 hour Nitro-Dur™ glyceryl trinitrate patch, Schering-Plough, Australia), and the other group performed tendon rehabilitation and used a placebo transdermal patch (one quarter of a Nitro-Dur™ demonstration patch). The active and placebo patches were indistinguishable from one another. The randomization was controlled by the senior pharmacist at the institution who also supervised the packaging of transdermal patches and their distribution to patients. Both the patients and the clinical examiner were blinded as to which group the patients were in (i.e., double-blind).

The transdermal patches were intact when distributed, and patients were required to cut the patches into quarters prior to application. Patients were also given a supply of paracetamol tablets (500 mg), and were instructed to use them exclusively for any headaches experienced.

Patients were instructed in the application of the patches at their initial visit. They were informed that the dosing regimen was one quarter of a transdermal patch to be applied daily to the skin area closest to the affected tendon. The patches were to be left in situ for 24 hours and then replaced with a new quarter patch. The site of application was demonstrated as over the site of maximal tendon tenderness (region 2 to 6 centimeters from the calcaneal insertion of the Achilles tendon; immediately distal to the lateral humeral epicondyle; and immediately distal to the anteroinferior aspect of the acromion). Patients were instructed to rotate the patch application site around this point with each new patch application for the six-month study duration in an effort to minimize application site irritation.

At the initial clinical assessment, all patients were instructed in the performance of a tendon specific rehabilitation program. The aim of this program was to encompass the current non-operative management for tendinopathy, and involved the following regimens. Rehabilitation for Achilles tendon was as follows: (a) rest from aggravating activities in the early stages (particularly repetitive weight-bearing activities such as walking, running, and jumping), (b) the use of 1-1.5 centimeter heel raises, (c) prolonged daily static stretching of the gastrocnemius and soleus musculature, and (d) an eccentric calf muscle strengthening program. Rehabilitation for the extensor carpi radialis brevis tendon was as follows; (a) rest from aggravating activities in the early stages (particularly strong gripping and repetitive forearm and wrist movements), (b) the early continuous use of a forearm counterforce brace, (c) prolonged daily static stretching of the wrist extensor musculature, and (d) a muscle strengthening program initially using isometric exercise and progressing to isotonic exercises of both concentric and eccentric types. For the supraspinatus and rotator cuff tendons, rehabilitation was as follows: (a) early rest from aggravating activities (especially heavy lifting, overhead and behind the back activities), (b) daily range of motion exercises and stretching of the posterior shoulder capsule and pectoral muscles, and (c) muscle strengthening with scapular retraction exercises and closed kinetic chain isometric exercises, gradually progressing to dynamic open kinetic chain isotonic resistance exercises.

In addition, at the initial visit and at all subsequent visits, the patient was required to complete a tendon specific symptom assessment sheet using verbal descriptor scales to rate the severity (0-4: none, mild, moderate severe, very severe) of their tendon pain with activity, at rest, and at night. This verbal descriptor questionnaire has been validated as a reliable measure of monitoring pain that is responsive to clinical change, and these three patient-rated pain scores were used as trial outcome measures.

Outcome measures. A single examiner assessed all patients and recorded information on clinical outcome measures. All clinical assessments were repeated at week 0, 2, 6, 12, and 24 with an identical format. Records of headaches, paracetamol use, and compliance with patch application and the tendon rehabilitation program were also made at these scheduled visits. Patients were excluded from the trials for non-compliance at any two visits.

For the Achilles tendinopathy trial the outcome measures were as follows: (a) the degree of Achilles tendon tenderness, as assessed using a four point scale (0-3: none, mild, moderate, severe tenderness), (b) patient-rated analogue pain score after the single leg stationary 10 hop test (rated 0-10), (c) measurement of ankle plantarflexor mean peak force (in Newtons) using a resisted footplate device, and (d) measurement of total ankle plantarflexor work using the ORI-ASTS (in Newtons per 20 seconds). This valid and reliable resisted footplate test involved seating the patient with the foot secured to the footplate, and required them to perform a 20 second effort of repeated ankle plantarflexion and dorsiflexion. The footplate was linked to a load cell and the readings were stored directly on computer hard drive using LabView 5.1 biomechanical software (National Instruments, California, U.S.A.).

For the extensor tendinopathy trial the clinical outcome measures were as follows: (a) assessment the level of local epicondylar and proximal common extensor tendon tenderness using a 4 point scale (0-3: none, mild, moderate, severe tenderness), (b) hand-held dynamometer measurement of resisted 3rd finger metacarpophalangeal extension with a fully extended elbow (in Newtons), (c) measurement of wrist extensor tendon mean peak force (in Newtons) using a modified chair pick-up test, and (d) measurement of total work using the ORI-TETS (in Newtons per 10 seconds). This modified chair pick up test has demonstrated reliability and validity for testing extensor tendinopathy patients, and was performed with the elbow flexed to ninety degrees, and a vertically oriented hand board gripped palm downwards and pulled superiorly for a maximal 10 second effort. The hand board was linked in series with a load cell and the readings stored directly on computer hard drive using LabView 5.1 biomechanical software (National Instruments, California, U.S.A.).

For the supraspinatus tendinopathy trial the clinical outcome measures were as follows: (a) assessment of anteroinferior subacromial tenderness (0-3: no tenderness, mild, moderate, severe), (b) visually assessed passive shoulder range of motion in abduction, forward flexion, external rotation (in degrees), and internal rotation (hand behind back; in centimetres from vertebra prominens), (c) hand-held dynamometer measurement of muscle force in “empty can” position (90 degrees abduction in scapular plane with full internal rotation), adduction, external rotation, internal rotation, and subscapularis push-off (in Newtons), and

(d) impingement tests in internal rotation (Hawkins test) and external rotation (0-1: negative or positive).

Outcome measures were analyzed with Sigmastat 2.0 statistical software (Jandel Scientific, California, U.S.A) using Mann-Whitney rank sum tests to compare differences between groups, and using the Wilcoxon sign rank test to compare differences within the groups. The level of significance was defined at p=0.05. A Chi square analysis of patient reported symptom outcomes at week 24 was performed. Effect size estimates were calculated by dividing the mean z-score, calculated from all outcome measures at week 24, by the square root of the sample size to given a general measure of the overall effect of the patch on pain, tendon force and function.

Analysis of the clinical trial outcome measures for all three trials determined that the data was not normally distributed. Mann-Whitney rank sum analysis compared the glyceryl trinitrate groups with the placebo groups for the individual specific tendinopathies. The significant results are summarized in Table I.

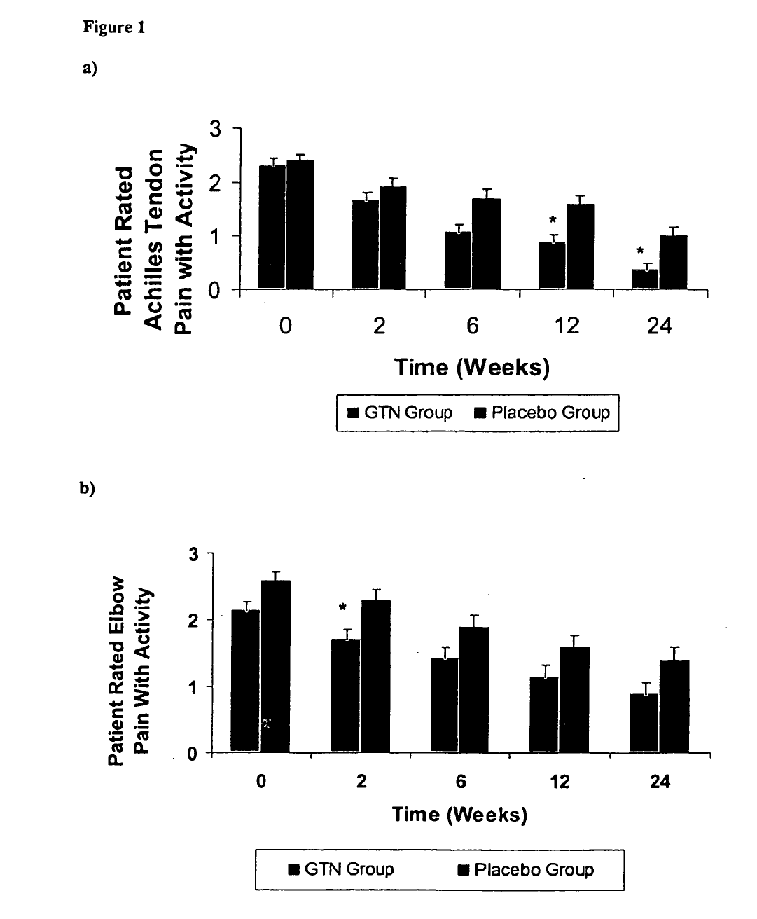

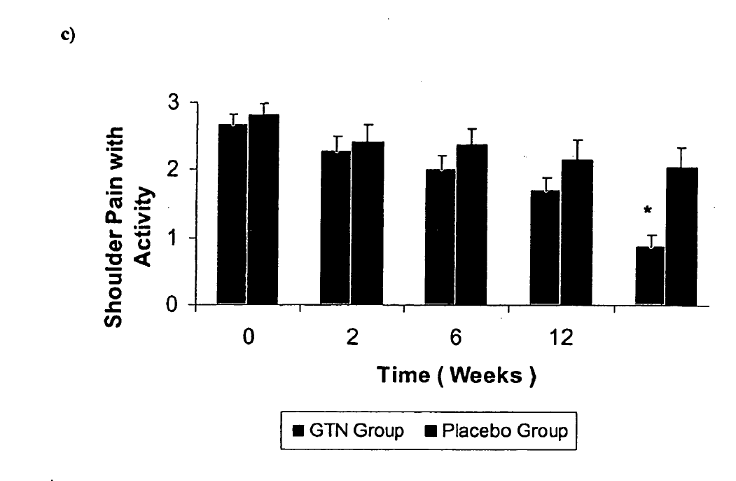

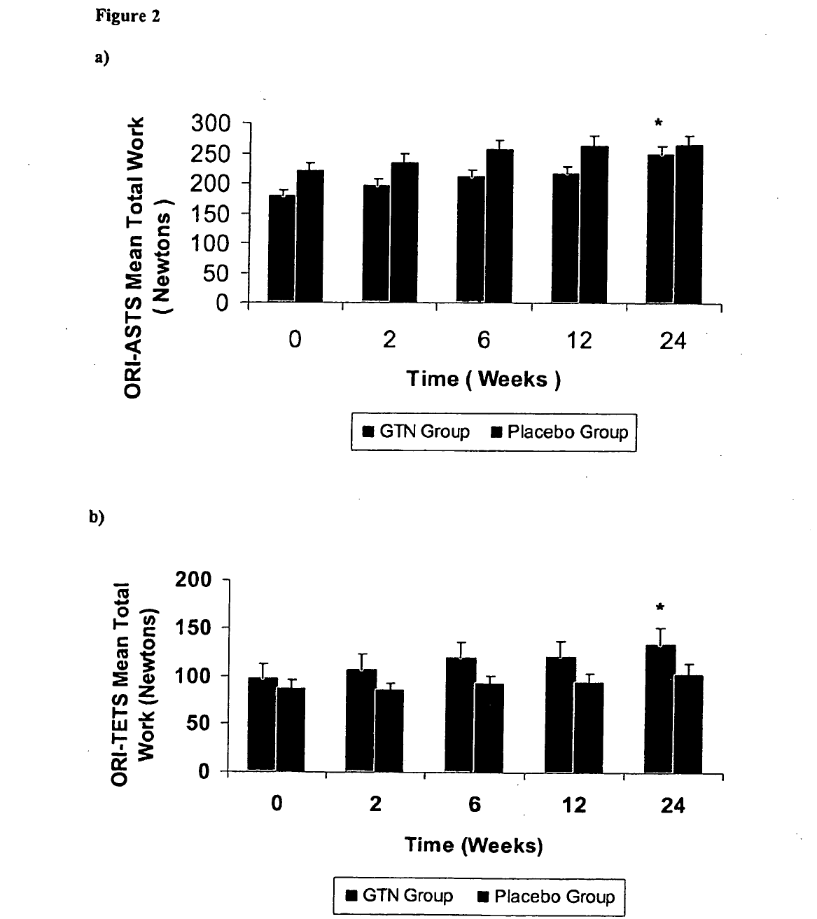

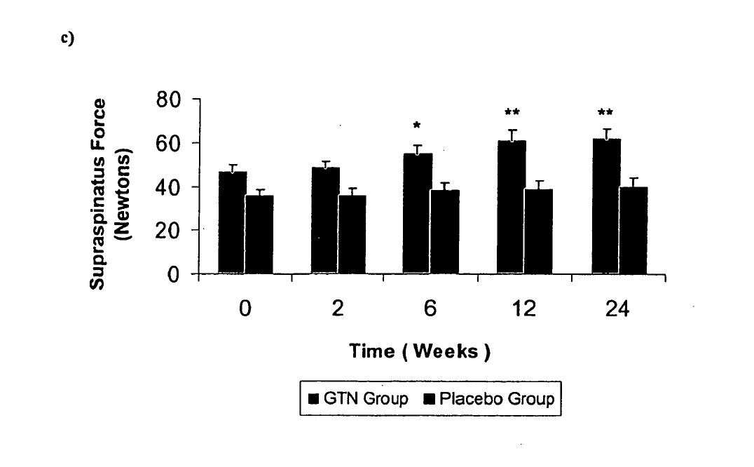

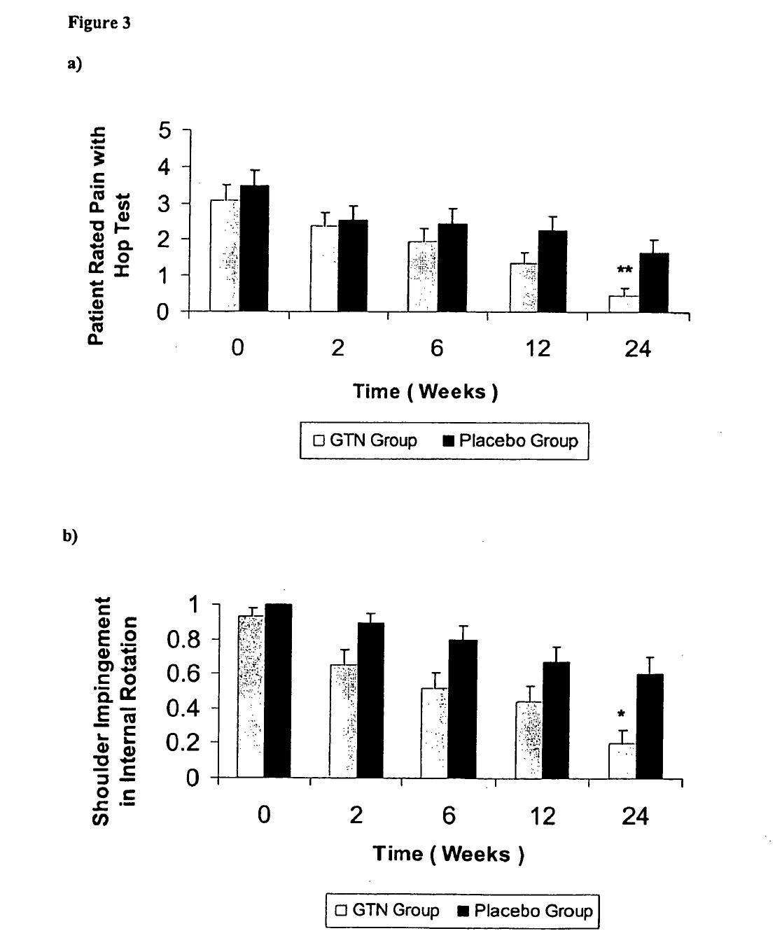

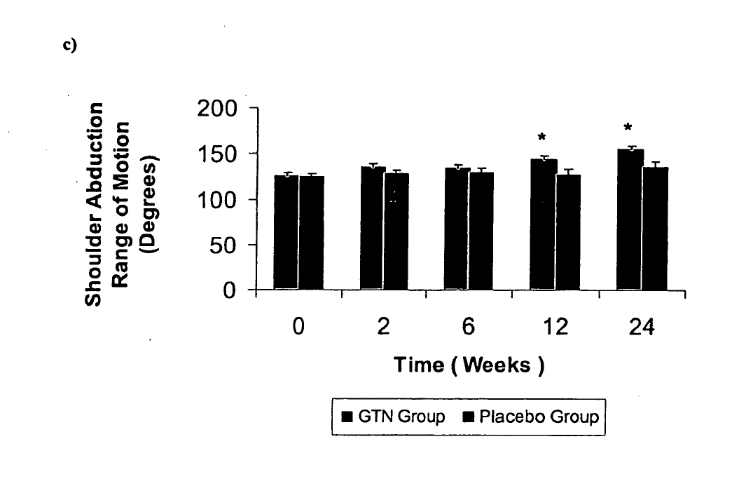

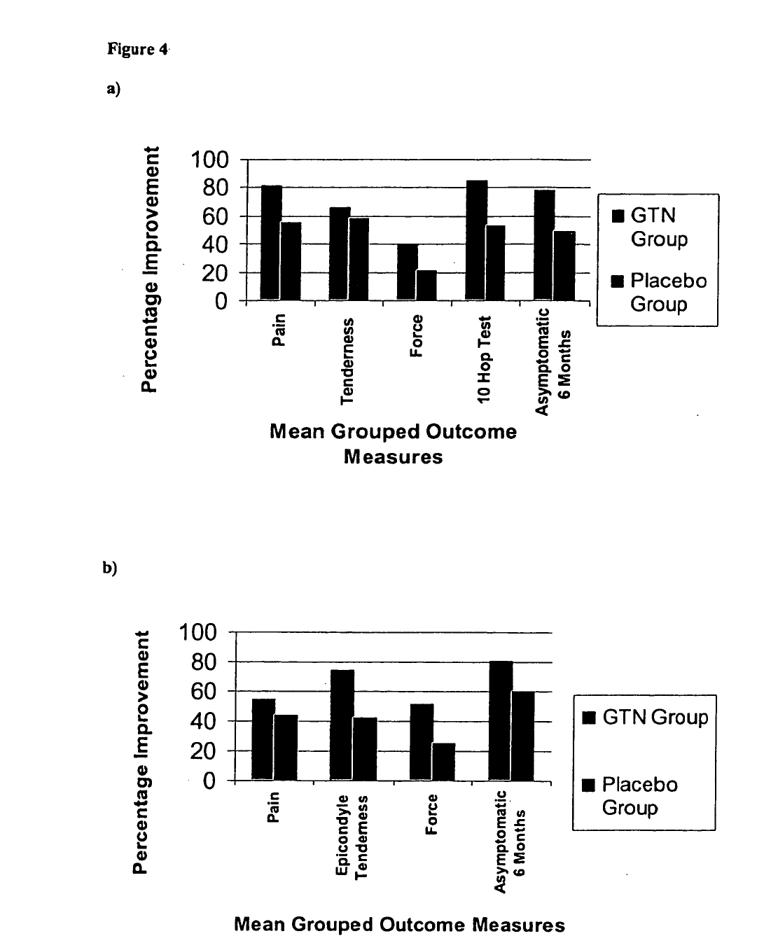

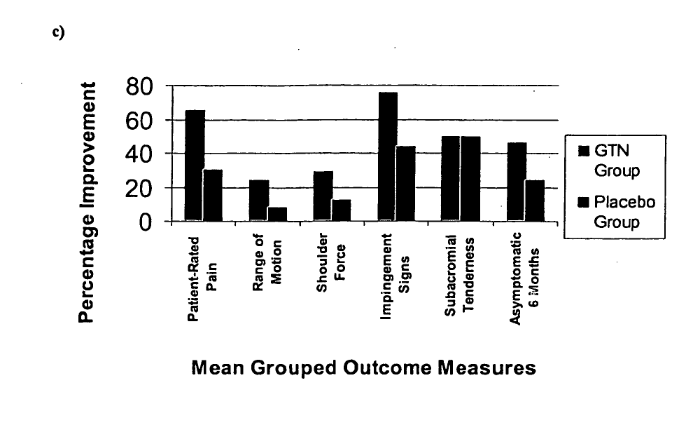

Pain. Pain outcome measures in the non-insertional Achilles tendinopathy trial demonstrated that the glyceryl trinitrate group compared to the placebo group had a significant decrease in Achilles tendon pain with activity at week 12 (p=0.02) and at week 24 (p=0.03) (FIG. 1a), and a significant decrease in night pain at week 12 (p=0.04). Pain outcome measures in the extensor tendinopathy trial the glyceryl trinitrate group also showed a significant decrease in elbow pain with activity at week 2 when compared to the placebo group (p=0.01) (FIG. 1b). Pain outcome measures in the supraspinatus tendinopathy trial similarly showed that the glyceryl trinitrate group compared to the placebo group had a significant decrease in shoulder pain with activity at week 24 (p=0.01) (FIG. 1c), a significant decrease in night pain at week 12 (p=0.03) and at week 24 (p=0.01), and a significant decrease in rest pain at week 12 (p=0.04) and week 24 (p=0.03).

Mann-Whitney rank sum tests comparing tendon tenderness between groups in the clinical trials showed significantly less Achilles tenderness at week 12 (p=0.02), and significantly less lateral epicondylar tenderness at week 6 (p=0.02) and at week 12 (p=0.02), in the glyceryl trinitrate group.

TABLE I

| | | Achiles | | Shoulder |
|---|---|---|---|---|
| Trial Parameters | | (N = 65 | Elbow (N = 86) | (N = 53) |
| Improved Patient | | 29% | 21% | 22% |
| Outcomes | | | | |
| Effect Size | | 0.14 | 0.12 | 0.26 |
| Pain | Activity | Decreased | Decreased | Decreased |
| Outcomes | | Week | Week 2 | Week 24 |
| | | 12/24 | | |
| | Night | Decreased | — | Decreased |
| | | Week 12 | | Week 12/24 |
| | Rest | — | — | Decreased |
| | | | | Week 12/24 |

| | | | |
|---|---|---|---|
| Force Outcomes | Increased | Increased Mean | Increased |
| | Mean | peak force and | supraspinatus, |
| | Total | mean total work | ER, IR, adduction |
| | Work | Week 24 | subscapularis |
| | Week 24 | | Week 12/24 |
| Tenderness | Decreased | Decreased Week | — |
| Outcomes | Week 12 | 6/12 | |
| Functional | Increased | — | Increased |
| Outcomes | Hop Test | | abduction, IR |
| Measures | Week 24 | | ROM Week 24, |
| | | | Decreased IR |
| | | | Impingement |
| | | | Week 24 |

Table I: Summarized results of the topical glyceryl trinitrate clinical trials on Achilles tendinopathy, extensor tendinopathy at the elbow, and supraspinatus tendinopathy. Includes patient outcomes, effect sizes, and demonstrated significant differences in trial outcome measures.

Force outcome. Regarding force outcome measures, the glyceryl trinitrate group compared to the placebo group in the non-insertional Achilles tendinopathy trial had a significant increase in ORI-ASTS measured mean plantarflexion total work from baseline levels at week 24 (p=0.04) (FIG. 2a), and in the extensor tendinopathy trial had a significant increase in ORI-TETS measured mean peak force at week 24 (p=0.03) and a significant increase in ORI-TETS mean total work at week 24 (p=0.03) (FIG. 2b). In the supraspinatus tendinopathy trial, the glyceryl trinitrate group had significantly increased supraspinatus force at week 6 (p=0.01), week 12 (p=0.001) and week 24 (p=0.001) (see FIG. 2c), significantly increased external rotation force at week 12 (p=0.01) and week 24 (p=0.004), significantly increased internal rotation force at week 12 (p=0.01) and week 24 (p=0.01), significantly increased subscapularis force at week 2 (p=0.01), week 12 (p=0.02) and week 24 (p=0.01), and significantly increased adduction force at week 12 (p=0.01) and week 24 (p=0.04).

Functional outcome. The glyceryl trinitrate group compared to the placebo group in the non-insertional Achilles tendinopathy trial also had a significant decrease in pain scores after the 10 hop test at week 24 (p=0.005) (FIG. 3a) in regard to functional outcome measures, and in the supraspinatus tendinopathy trial had a significant decrease in impingement in internal rotation at week 24 (p=0.02) (FIG. 3b), a significant increase in passive shoulder abduction range of motion at week 12 (p=0.03) and week 24 (p=0.02) (FIG. 3c), and a significant increase in shoulder internal rotation range of motion at week 24 (p=0.04).

In the Achilles tendinopathy trial patient reported outcomes at week 24 showed that 78% of patients in the glyceryl trinitrate group had excellent improvement (asymptomatic with activities of daily living) over the course of the trial compared with patient ratings of 49% excellent in the placebo group (FIG. 4a). In the extensor tendinopathy trial patient reported outcomes at week 24 showed that 81% of patients in the glyceryl trinitrate group had excellent improvement over the course of the trial compared with patient ratings of 60% excellent in the placebo group (FIG. 4b). In the supraspinatus tendinopathy trial patient reported outcomes at week 24 showed that 46% of patients in the glyceryl trinitrate group had excellent improvement over the course of the trial compared with patient ratings of 24% excellent in the placebo group (FIG. 4c). Chi square analyses comparing outcomes between the two groups revealed that the glyceryl trinitrate group had a significantly increased (p=0.001) chance of being asymptomatic with activities of daily living at 24 weeks in all three clinical trials (Achilles tendinopathy trial: p=0.001, number needed to treat (NNT)=3.4), (extensor tend inopathy trial: p=0.005, NNT=4.8), (supraspinatus tend inopathy trial: p=0.007, NNT=4.5).

Effect size estimations at week 24 in the three clinical trials were for glyceryl trinitrate in the treatment of Achilles tendinopathy 0.14 (95% CI 0.09-0.19), for glyceryl trinitrate in the treatment of extensor tendinopathy at the elbow 0.12 (95% CI 0.06-0.19), and for glyceryl trinitrate in the treatment of supraspinatus tendinopathy 0.26 (95% CI 0.19-0.32).

In the clinical trials the majority of patients in the glyceryl trinitrate group experienced headache as a side-effect (Table II), however, only in the supraspinatus tendinopathy trial was there a significant increase in the number of days affected by headache (p=0.001). There were significant increases in the total amount of paracetamol required for headache treatment in the glyceryl trinitrate group for the Achilles tendinopathy trial (p=0.001), and the supraspinatus tendinopathy trial (p=0.001).

Within the three clinical trials there were no significant differences between groups in drop-out rates or trial completion rates (Table II). The patients that were discontinued from the clinical trials, mainly for side-effects of headache or application site rash, were all receiving topical glyceryl trinitrate.

TABLE II

| | | Achilles | | Shoulder |
|---|---|---|---|---|
| Trial Parameters | | (N = 65) | Elbow (N = 86) | (N = 53) |
| Trial Completion Rate | | GTN 84% | GTN 81% | GTN 88% |
| | | Placebo 94% | Placebo 91% | Placebo 93% |
| Discontinuations (All | | Rash, | Rash 2, | Headache 2 |
| GTn Group) | | Headache 1 | Headache 2 | |
| Drop-outs | | GTN 2 | GTN 3 | GTN 1 |
| | | Placebo 1 | Placebo 4 | Placebo 2 |
| Headache | Total | GTN 85 (53%) | GTN 136 (63%) | GTN 127 |
| (Days) | | | | (76%) |
| | | Placebo 101 | Placebo 166 | Placebo 37 |
| | | (45%) | (58%) | (33%) |
| | Average | GTN 5 | GTN 5 | GTN 6 |
| | | Placebo 7 | Placebo 7 | Placebo 74 |
| | Median | GTN 4 | GTN 3 | GTN 4 |
| | | Placebo 3 | Placebo 1 | Placebo 0 |
| Paracetamol | Total | GTN 237 | GTN 214 | GTN 138 |
| | | Placebo 46 | Placebo 250 | Placebo 69 |
| | Average | GTN 14 | GTN 8 | GTN 7 |
| | | Placebo 3 | Placebo 10 | Placebo 8 |
| | Median | GTN 10 | GTN 4 | GTN 2 |
| | | Placebo 0 | Placebo 0 | Placebo 0 |
| Other Noted | GTN | Rash 16%, | Rash 21%, | Rash 8% |
| Side-Effects | | Increase | Increase Axillary | |
| | | Tinnitus (3%) | Sweating 2% | |
| | Placebo | Rash 12% | Rash 9% | Rash 7% |

| | | | |
|---|---|---|---|
| No Side Effects | GTN 44% | GTN 35% | GTN 30% |
| | Placebo 45% | Placebo 33% | Placebo 59% |

Table II: Summarized results of the topical glyceryl trinitrate clinical trials on Achilles tendinopathy, extensor tendinopathy at the elbow, and supraspinatus tendinopathy. Includes trial completion rates, discontinuations, drop-outs, and noted side-effects.

These three randomized, double blind, placebo controlled clinical trials demonstrate that continuous 1.25 mg/24 hour topical glyceryl trinitrate application used as therapy for chronic tendinopathies can result in significantly decreased tendon pain with activity, significantly decreased tendon tenderness, significantly improved functional measures, and significantly improved patient outcomes when compared with tendon rehabilitation alone.

At the completion of the clinical trials 21-29% more patients in the glyceryl trinitrate-treated group than the placebo group were asymptomatic with activities of daily living, and rated their specific tendon as excellent. From these results the number of patients needed to treat (NNT) to obtain a positive outcome can be calculated. For every 3.4 chronic Achilles tendinopathy patients, every 4.8 extensor tendinopathy patients, and every 4.5 supraspinatus tendinopathy patients treated with topical glyceryl trinitrate therapy, one patient will have an excellent result at 24 weeks that would not have occurred with placebo treatment.

The mean estimated effect sizes at week 24 for the three clinical trials ranged from 0.12-0.26, which are equivalent to binomial effect size displays, or changes in patient success rates of 12-26%. This effect size range is comparable to the 21-29% improvement in patient rated outcomes noted with topical glyceryl trinitrate therapy. These closely related parallel outcomes calculated from very different sources (patient rated outcomes versus all trial outcome measures) apparently quantify the estimated size of the effect of topical glyceryl trinitrate in treating chronic tendinopathies. While the overall outcomes from the three clinical trials appear closely related, the individual outcome measures require a closer analysis to determine the effects of topical glyceryl trinitrate on tendons.

Within the clinical trials the outcome measure of tendon pain with activity was significantly improved in the glyceryl trinitrate groups in all three trials, although the timing of the improvement varied from early in extensor tendinopathy, to late with non-insertional Achilles tendinopathy and supraspinatus tendinopathy. The reason for this may be due to the immediately subcutaneous position of the lateral humeral epicondyle and extensor carpi radialis brevis tendon. Despite the fact that the Achilles tendon is also subcutaneous, it is less regular in contour (especially with any variation in patch application to either the medial or lateral aspect of the tendon).

An analysis of the between group means at week 0 compared with week 24 demonstrated that the glyceryl trinitrate group patient-rated pain scores (with activity, at night, and at rest) for the trials decreased by an average of 65% (range 64-67%), while the placebo group scores for the trials decreased by an average of 30% (range 27-33%) (FIGS. 4a-c). These results suggest that topical glyceryl trinitrate may have a pain modulation effect in chronic tendinopathies, although the effect appears to differ in timing between specific tendon sites. Possible mechanisms for this effect include increased blood supply to the region due to local vasodilatation, increased clearance of local inflammatory mediators or bioactive proteins such as substance P, or local effects on neural structures, neovascularisation, or apoptosis that may lead to modulation of tendon pain.

Across all three clinical trials there were significant increases in force outcome measures in the glyceryl trinitrate groups at the week 24 stage, with the Orthopaedic Research Institute-Ankle Strength Testing System (ORI-ASTS) and Tennis Elbow Testing System (ORI-TETS), demonstrating increased mean total work, and all dynamometer resisted force measurements for the rotator cuff tendons demonstrating significant increases. These outcome measures have demonstrated excellent intra-rater reliability and validity in testing patients with specific chronic tendinopathies. An analysis of the between group means at week 0 compared with week 24 demonstrated that the glyceryl trinitrate group force outcome measures for the trials increased by an average of 37% (range 33-38%), while the placebo group scores for the trials increased by an average of 16% (range 11-20%). These results suggest that topical glyceryl trinitrate may have an effect on tendon that increases force measures in chronic tendinopathies. This may be a direct effect on tendon metabolism or fibroblasts possibly increasing collagen synthesis and remodeling or an indirect effect due to possible pain modulation.

In the glyceryl trinitrate groups functional outcome measures were significantly increased at week 24 relative to the placebo group in all three clinical trials. These functional tests included the 10 hop test for non-insertional Achilles tendinopathy, the ORI-TETS mean peak force and mean total work for extensor tendinopathy, and shoulder passive range of motion in abduction and in internal rotation, as well as shoulder impingement in internal rotation for supraspinatus tendinopathy. All of these measures reflect important functional characteristics of the tendons involved: hopping involves Achilles tendon loading through push-off and landing as used in running and jumping; wrist extensor tendon peak force and total work measured with a modified chair pick-up test (ORI-TETS) as seen when lifting heavy objects; shoulder range of motion in abduction when utilizing supraspinatus function for overhead activities, shoulder range of motion in internal rotation as used with toileting and dressing, and shoulder impingement in internal rotation which is a common cause of shoulder pain in patients with supraspinatus tendinopathy and may perpetuate the “vicious cycle” of rotator cuff tendon injury and dysfunction. These results indicate that glyceryl trinitrate may modulate tendon function, and again this may be through direct or indirect effects on tendon, but correlates with the results of both decreased pain and increased force suggesting increased control of movement.

Clinical assessment of tendon tenderness revealed significant decreases in the glyceryl trinitrate groups at week 12 in both the Achilles and elbow tendinopathy clinical trials. There were no significant differences in the supraspinatus tendinopathy trial. These results may be due to the subcutaneous nature of the Achilles and extensor carpi radialis brevis tendons relative to the deeper supraspinatus tendon. The decreased tenderness precedes any significant improvements in force and function measurements (and may represent a manifestation of pain modulation prior to any structural alteration in tendon allowing increased force production.)

The number of patients discontinued during the course of the clinical trials ranged from 4-6% of clinical trial patients, these patients were all in the glyceryl trinitrate groups, and they were discontinued for recognized side-effects of headache or application site rash. One patient was discontinued for recurrent facial flushing, which was reversible on discontinuation of the medication. This patient was a type 2 diabetic and it was felt that this side-effect was caused by arteriolar dilatation (Table II).

The trial completion rate for the glyceryl trinitrate group ranged from 81-88% and the placebo group ranged from 91-94%. There was no significant difference between groups in regard to completion, or drop-out, rates between groups. If discontinued patients were excluded from this analysis, the trial completion rates differed by less than 4%. The high completion rate amongst groups may be due to the thorough explanation of requirements for the clinical trial prior to entry, frequent assessment visits, relatively low side-effect profile of the medication, or the personalities of patients entering clinical trials.

Headache was the most frequent side-effect and in the glyceryl trinitrate group and ranged from 53-76% of patients, with an average number of days of headache ranging from 5-6 days, and the median number of days of headache ranging from 3-4 days. 72% of headaches in the glyceryl trinitrate groups occurred within the first two weeks of the trial. The percentage of patients experiencing headache in these clinical trials was higher than that reported in the literature of 18-68% for dosages of 5 mg/24 hour. It is difficult to understand the reasons for this, especially as the dosing regime used in the clinical trials was a continuous low dose of 1.25 mg-2.5 mg/24 hours, but this may be due to better patient reporting of side-effects, since patients were required to complete a headache diary which was checked for compliance. The placebo groups also reported high rates of headache ranging from 33-58% of patients, with an average number of days of headache ranging from 4-7 days, and the median number of days of headache ranging from 0-3 days.

Patients in the clinical trials were supplied with paracetamol (Tylenol™) tablets for exclusive use with potential headaches. In the glyceryl trinitrate groups the total paracetamol usage ranged from 138-237 tablets, with an average of 7-14 tablets, and a median of 2-10 tablets. In the placebo groups the total paracetamol usage ranged from 69-250 tablets, with an average of 3-10 tablets, and a median of 0 tablets. There were significant between-group differences in the reported rate of headache and the average number of headaches experienced in the supraspinatus tendinopathy trial, but not in the other clinical trials. There were also significant between-group differences in the total amount of paracetamol used in the Achilles tendinopathy and supraspinatus tendinopathy trials. The higher rates of headache in the supraspinatus tendinopathy trial may be due to the glyceryl trinitrate patch application site being closer to both the cardiac and cerebral circulation than either the extensor tendinopathy or Achilles tendinopathy trials, possibly leading to greater systemic and local vasodilation. Despite the high rates of headache in the supraspinatus tendinopathy trial, the use of paracetamol was lower than in either of the other clinical trials. It should be noted that, in general, the glyceryl trinitrate group experienced more severe headaches than the placebo group, as evidenced by 1-2 patients in each clinical trial discontinued due to this side-effect and the placebo group median use of paracetamol being zero.

Another common side-effect of topical glyceryl trinitrate was application site rash and in the glyceryl trinitrate groups the number of patients experiencing rash ranged from 8-21%. This compared with rates in the placebo groups ranging from 7-12%. Reports in the literature for glyceryl trinitrate dosages of 5 mg/24 hour note rash occurred in 16-38% of patients, and these side-effect rates are comparable with those reported in these clinical trials. There was a greater severity of rash in the glyceryl trinitrate groups compared to the placebo groups as evidenced by a total of five patients discontinued due to this side-effect.

Other side-effects that were reported included: an increase in pre-existing tinnitus, increased ipsilateral axillary sweating, and a perception of apprehension. None of these were severe, and all were reversible on discontinuation of the medication at the conclusion of the clinical trials. The number of patients in the glyceryl trinitrate groups that experienced no side-effects ranged from 30-44%, while those in the placebo groups ranged from 33-59%.

These clinical trials investigating topical glyceryl trinitrate donation with tendon rehabilitation demonstrated improved patient rated pain scores, increased tendon force measures, improved functional measures, and improved patient outcomes relative to tendon rehabilitation alone in the treatment of chronic overuse tendinopathies. Topical NO donors such as 1.25 mg/24 hour glyceryl trinitrate have a long history of therapeutic use in humans, have a known side-effect profile with no irreversible effects, and now have clinically demonstrated efficacy in modulating pain, force measures, functional measures, and patient outcomes at six months in specific chronic overuse tendinopathies. These studies it establish that transdermal glyceryl trinitrate is effective in treating specific overuse tendinopathies in mammals and especially in humans.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

### Example 2

A 35 year old male patient suffering from chronic tendinopathy of the left Achilles tendon applies a transdermal patch delivering 0.03 mcg/hr nitroglycerin for a period of two weeks. The patient experiences a moderate decrease in tenderness and ankle soreness by day 2 of therapy, which progressively improves over the treatment period. Following the treatment period, the patient feels his ankle is pain free. The ankle remains pain free for several weeks beyond the treatment period.

### Example 3

A 35 year old female patient suffering de Quervain's tendinopathy in the right extensor tendons of the thumb applies a transdermal patch delivering 0.01 mcg/hr nitroglycerin for a period of four weeks. This patient suffers this condition due to the arrival of a new baby and the consequent carrying as an unusual daily activity, and physical therapy and intermittent use of a wrist splint provides little relief of symptoms. The patient notices a decrease in pain within one day of beginning treatment, and a subsequent assessment by a physician at week four of treatment reveals no positive signs or symptoms of de Quervain's disease. This includes a negative Finklestein test. The patient remains pain free for several months post-treatment.

### Example 4

A 65 year old male patient suffering from chronic tennis elbow applies a transdermal patch delivering 0.06 mcg/hr nitroglycerin for a period of one week. The patient experiences a moderate decrease in pain upon elicitation at the end of the treatment period as assessed by grip strength and resisted wrist dorsiflexion. The patient remains with some residual symptoms, though the patient's symptoms are less severe than before treatment.

What is claimed is:

1. A method of treating tendinopathy in the absence of inflammation comprising administering to a mammal in need of such treatment a glyceryl trinitrate containing transdermal patch, said transdermal patch being administered to a skin site proximate an affected tendon and being configured to deliver glyceryl trinitrate at a rate of from about 5 mcg/hr to about 85 mcg/hr.

2. The method of claim 1, wherein the transdermal patch is configured to deliver glyceryl trinitrate at a rate of from about 15 mcg/hr to about 75 mcg/hr.

3. The method of claim 1, wherein the transdermal patch is configured to deliver glyceryl trinitrate at a rate of from about 30 mcg/hr to about 65 mcg/hr.

4. The method of claim 1, wherein the tendionopathy is chronic.

5. The method of claim 1, wherein the tendinopathy is tendinosis.

6. The method of claim 1, wherein the tendinopathy is overuse tendinopathy.

7. The method of claim 1, wherein the tendopathy is tendonitis.

8. The method of claim 1, wherein the tendinopathy is extensor tendinopathy at the elbow.

9. The method of claim 1, wherein the tendinopathy is Achilles tendinopathy.

10. The method of claim 1, wherein the tendinopathy is supraspinatus tendinopathy.

11. The method of claim 1, wherein the mammal is a human.

12. The method of claim 1, wherein the tendinopathy is selected from the group consisting of patellar tendinopathy, quadriceps tendinopathy, hip adductor tendinopathy, common flexor tendinopathy of the elbow, and tendinopathy of the thumb.

13. The method of claim 1, which comprises continuously administering the glyceryl trinitrate for between about 1 and about 24 weeks.

14. The method of claim 1, which comprises continuously administering the glyceryl trinitrate for between about 12 and about 24 weeks.

15. The method of claim 1, wherein the wherein the glyceryl trinitrate is continuously released over a pre-determined period of time of about 24 hours.

16. The method of claim 1, which further comprises treating the mammal with a non-operative rehabilitation regimen comprising at least one of rest, tendon unloading, orthotics, braces, daily prolonged static stretching, or a graduated strengthening exercise program comprising eccentric tendon loading, or combinations thereof, during at least a portion of the time that the mammal is administered the glyceryl trinitrate.

17. A method of relieving pain caused by tendinopathy in the absence of inflammation comprising administering a transdermal patch containing glycerol nitrate to a mammal in need thereof, said transdermal patch being applied to a skin site proximate an affected tendon and being configured to deliver glyceryl trinitrate at a rate of from about 5 mcg/hr to about 85 mcg/hr, wherein the administration is for a length of time wherein pain of the affected tendon is relieved.

18. The method of claim 17, wherein the transdermal patch is configured to deliver from about 15 mcg/hr to about 75 mcg/hr.

19. The method of claim 17, wherein the transdermal patch is configured to deliver from about 30 mcg/hr to about 65 mcg/hr.

20. The method of claim 17, wherein the tendinopathy is chronic.

21. The method of claim 17, wherein the tendinopathy is tendinosis.

22. The method of claim 17, wherein the tendinopathy is overuse tendinopathy.

23. The method of claim 17, wherein the tendopathy is tendonitis.

24. The method of claim 17, wherein the tendinopathy is extensor tendinopathy at the elbow.

25. The method of claim 17, wherein the tendinopathy is Achilles tendinopathy.

26. The method of claim 17, wherein the tendinopathy is supraspinatus tendinopathy.

27. The method of claim 17, wherein the mammal is a human.

28. The method of claim 17, wherein the tendinopathy is selected from the group consisting of patellar tendinopathy, quadriceps tendinopathy, hip adductor tendinopathy, common flexor tendinopathy of the elbow, and tendinopathy of the thumb.

29. The method of claim 17, which comprises continuously administering the glyceryl trinitrate for between about 1 to about 24 weeks.

30. The method of claim 29, which comprises continuously administering the glyceryl trinitrate for between about 2-24 weeks.

31. The method of claim 30, which comprises continuously administering the glyceryl trinitrate for between about 12-24 weeks.

32. The method of claim 24, wherein the relieving pain is a decrease in elbow pain with activity or a decrease in elbow tenderness, or a combination thereof.

33. The method of claim 25, wherein the relieving pain is a decrease in Achilles tendon pain with activity or a decrease in night pain, a decrease in Achilles tendon tenderness or a combination thereof.

34. The method of claim 26, wherein the relieving pain is a decrease in shoulder pain with activity or a decrease in night pain, a decrease in rest pain, a decrease in shoulder tenderness, or a combination thereof.

35. The method of claim 24, wherein the mammal is further treated by a non-operative rehabilitation regimen comprising at lease one of rest, tendon unloading, orthotics or braces, daily prolonged static stretching, or a graduated strengthening exercise program comprising eccentric tendon loading, or combinations thereof, during at least a portion of the time that the mammal is treated with glyceryl trinitrate.

36. A method of reducing tenderness related to tendinopathy in the absence of inflammation comprising administering a transdermal patch containing glycerol nitrate to a mammal in need thereof, said transdermal patch being applied to a to a skin site proximate an affected tendon and being configured to deliver glyceryl trinitrate at a rate of from about 5 mcg/hr to about 85 mcg/hr, wherein the administration is for a length of time such that tenderness of the affected tendon is reduced.

37. The method of claim 36, wherein the transdermal patch is removed and a new patch is applied to a new skin site proximate the affected tendon.

38. The method of claim 36, wherein the tendinopathy is tendinosis.

39. The method of claim 36, wherein the tendinopathy is overuse tendinopathy.

40. The method of claim 36, wherein the tendopathy is tendonitis.

41. A method of treating tendinopathy in the absence of inflammation comprising administering a transdermal patch containing glycerol nitrate to a mammal in need thereof, said transdermal patch being applied to a skin site proximate an affected tendon and being configured to deliver glyceryl trinitrate at a rate of from about 5 mcg/hr to about 85 mcg/hr, and wherein the administration is for a length of time such that function of the affected tendon is improved.

42. The method of claim 41, wherein the tendinopathy is tendinosis.

43. The method of claim 41, wherein the tendinopathy is overuse tendinopathy.

44. The method of claim 41, wherein the tendopathy is tendonitis.

45. The method of claim 41, wherein the tendinopathy is extensor tendinopathy and wherein the improved function of the affected tendon i) provides for an improvement in mean peak force ii) provides for an improvement in mean total force iii) makes the mammal asymptomatic with activities of daily living, or iv) combinations thereof.

46. The method of claim 41, wherein the overuse tendinopathy is Achilles tendonopathy and wherein the improved function of the affected tendon i) provides for an increase in mean plantarflexion total work force ii) makes the mammal asymptomatic with activities of daily living, or iii) a combination thereof.

47. The method of claim 41, wherein overuse tendonopathy is supraspinatus tendinopathy and wherein the improved function of the affected tendon i) provides for an increase in external rotation force, ii) provides for an increase in subscaplularis force, iii) provides for an increase in adduction force, iv) provides for an increase in internal shoulder rotation range, v) provides for an increase in passive shoulder abduction range of motion, vi) provides for a decrease in impingement in internal rotation, vii) makes the mammal asymptomatic with activities of daily living, or viii) combinations thereof.

48. A method of treating tendinopathy in the absence of inflammation comprising administering a transdermal patch containing glycerol nitrate to a mammal in need thereof, said transdermal patch being applied to a skin site proximate an affected tendon and being configured to deliver glyceryl trinitrate at a rate of from about 5 mcg/hr to about 85 mcg/hr, wherein the glyceryl trinitrate is continuously delivered, and wherein the function of the affected tendon is improved.

49. The method of claim 48, wherein the tendinopathy is tendinosis.

50. The method of claim 48, wherein the tendinopathy is overuse tendinopathy.

51. The method of claim 48, wherein the tendopathy is tendonitis

52. The method of claim 48, wherein said continuous administration of glyceryl trinitrate lasts for about 1 to about 24 weeks.

53. The method of claim 48, wherein said continuous administration of glyceryl trinitrate lasts for about 12 to about 24 weeks.

54. The method of claim 48, wherein the transdermal patch is removed and a new transdermal patch is applied to a new skin site.

55. A method of treating tendinopathy in the absence of inflammation comprising administering a transdermal patch containing glycerol nitrate to a mammal in need thereof, said transdermal patch being applied to a skin site proximate an affected tendon and being configured to deliver glyceryl trinitrate at a rate of from about 5 mcg/hr to about 85 mcg/hr, wherein the administration is such that there is decreased pain upon activity of the affected tendon.

56. The method of claim 55, wherein the tendinopathy is tendinosis.

57. The method of claim 55, wherein the tendinopathy is overuse tendinopathy.

58. The method of claim 55, wherein the tendopathy is tendonitis

59. A method of treating tendinopathy in the absence of inflammation comprising topically administering a composition containing glycerol nitrate to a mammal in need thereof, said composition being applied to a skin site proximate an affected tendon and comprising an effective amount of glyceryl trinitrate, wherein the glyceryl trinitrate is transdermally delivered over a pre-determined period of time, and wherein the administration is such that there is decreased night pain of the affected tendon.

60. The method of claim 59, wherein the tendinopathy is tendinosis.

61. The method of claim 59, wherein the tendinopathy is overuse tendinopathy.

62. The method of claim 59, wherein the tendopathy is tendonitis

63. A method of treating tendinopathy in the absence of inflammation comprising topically administering a composition containing glycerol nitrate to a mammal in need thereof, said composition being applied to a skin site proximate an affected tendon and comprising an effective amount of glyceryl trinitrate, wherein the glyceryl trinitrate is transdermally delivered over a pre-determined period of time, and wherein the administration is such that there is decreased amount of pain when the affected tendon is at rest.

64. The method of claim 63, wherein the tendinopathy is tendinosis.

65. The method of claim 63, wherein the tendinopathy is overuse tendinopathy.

66. The method of claim 63, wherein the tendopathy is tendonitis
